(19) United States
(12) Patent Application Publication
(10) Pub. No.: US 2005/0203710 A1
(43) Pub. Date: Sep. 15, 2005

(54) Parallel reaction executing apparatus, parallel reaction execution planning design apparatus, and parallel reaction executing method

(75) Inventors: Masami Hagiya, Tokyo (JP); Seika Abe, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignees: OLYMPUS CORPORATION, Tokyo (JP); Novusgene Inc., Tokyo (JP); Mitsui Knowledge Industry Co., Inc., Tokyo (JP)

(21) Appl. No.: 11/035,782

(22) Filed: Jan. 14, 2005

(30) Foreign Application Priority Data
Jan. 16, 2004 (JP) 2004-009626

(51) Int. Cl. G06F 19/00; G01N 33/48; G01N 33/50

(52) U.S. Cl. 702/19; 702/22

(57) ABSTRACT

A parallel reaction executing apparatus comprises an electronic computer and a parallel reaction executing section, the electronic computer substantially controlling a function of the parallel reaction executing section in addition to a current computing process, and executing a number of reactions in parallel under the control of the apparatus, wherein the parallel reaction executing section provided by the electronic computer controls parallel reactions in accordance with a program optimized after converting a higher level language produced in a predetermined language into a command of controlling the parallel reaction executing section.

### CROSS-REFERENCE TO RELATED APPLICATIONS

This application is based upon and claims the benefit of priority from prior Japanese Patent Application No. 2004-009626, filed Jan. 16, 2004, the entire contents of which are incorporated herein by reference.

### BACKGROUND OF THE INVENTION

1. Field of the Invention

The present invention relates to a novel parallel reaction executing apparatus, a parallel reaction execution planning design apparatus, and a parallel reaction executing method using a nuclear acid molecule.

2. Description of the Related Art

A DNA computer proposed by Adleman in 1994 is known, and a research of the computer is underway. This DNA computer is expected to compensate a conventional computer to a problem called “a Hamilton route problem” which is well known among mathematicians.

The DNA computer comprises: a molecular computer which causes an input and an output serving as a DNA to execute a reaction and which executes computation of a combination of DNAs for acquiring and detecting a result DNA; and an electronic computer which executes a processing program, outputs a result of the execution, and executes interpretation of a planning and a result of a calculation reaction.

In such a DNA computer, the molecular computer can execute a plurality of DNA reactions (hereinafter, simply referred to as “reactions”) in parallel. However, since these DNA reactions are executed under a variety of constraints, and require a large amount of time intervals, it is important to automatically and efficiently execute a plurality of reactions. Therefore, finite tables provided at the molecular computer must be allocated without wastefulness and commands requiring a large amount of time intervals such as a temperature control command must be efficiently scheduled.

With respect to this scheduling, for example, there is proposed an analyzing system which can make an input operation for an analyzing device easily, which is superior in general purpose usability, and moreover, which can replace and use analyzing equipment smoothly without any problem (refer to Jpn. Pat. Appln. KOKOKU Publication No. 7-92877). In the Jpn. Pat. Appln. KOKOKU Publication No. 7-92877, the analyzing device comprises an interpreter capable of translating an execution statement produced by a higher level language expression from a data processing device into command data for executing an operation. Thus, an understandable character string or the like which is close to a natural language inputted from the data processing device side, whereby printing, computation and the like can be freely carried out by setting parameters of the analyzing device and making key operation or by calling a parameter value and an actual measurement value. Accordingly, in the case of a direct operation, for example, in the case where an operator attempts to check a value of the analyzing device during operation, the operator can make operation immediately and simply, and operability is improved.

In addition, for example, there is proposed a method capable of possibly speedily processing a whole object targeted for inspection such as biological or morphological samples in a method of processing an object targeted for inspection, in particular, a method capable of processing cytological or morphological samples in an auto coloring device or a tissue processing device (refer to Jpn. Pat. Appln. KOKAI Publication No. 2002-90371). In this method, by a graph theoretical and/or linear planning based solution or by allocation or relocation of processing stations to be reached in preset order in order to execute each processing program, optimization of an amount of processing (or introduction of the best solution) is carried out by minimizing a traveling route of a conveying device. In this manner, as many objects targeted for inspection such as cytological or morphological samples as possible are conveyed within a processing time in each processing station so as to enable speedy processing through the whole process.

### BRIEF SUMMARY OF THE INVENTION

According to a first aspect of the present invention, there is provided a parallel reaction executing apparatus comprising an electronic computer and a parallel reaction executing section, the electronic computer substantially controlling a function of the parallel reaction executing section in addition to a current computing process, and executing a number of reactions in parallel under the control thereof, wherein control of the parallel reaction executing section by the electronic computer is executed in accordance with a program optimized after converting a higher level language produced in a predetermined language into a command of controlling the parallel reaction executing section.

According to a second aspect of the present invention, there is provided a parallel reaction executing apparatus for executing reactions in parallel in accordance with chemical reaction of a molecule, the apparatus comprising an electronic computer having: a parallel reaction executing section comprising a reaction control section which makes reaction control of chemical reaction of a molecule; a control command generator which converts a variable and a constant defined by associating the molecule with an encoded molecule that has been encoded in accordance with a nuclear acid sequence and a program described by a function defined to be associated with calculation reaction of the encoded molecule into a control command for driving the reaction control section which makes reaction control of chemical reaction of the molecule corresponding to the calculation reaction of the encoded molecule to generate procedures for the control command; and an output unit which outputs the procedures for the control command, wherein the control command generator converts the program produced in a predetermined language into the control command, and then optimizes the converted control command.

According to a third aspect of the present invention, there is provided a parallel reaction execution planning design apparatus for designing a parallel reaction planning for executing a number of reactions are executed in parallel, the apparatus comprising: a control command generator which converts a program described by a function defined to be associated with a reaction principle of parallel reactions into a control command for driving a reaction control section which makes reaction control of the parallel reactions, and generates procedures for the control command; and an output unit which outputs the procedures for the control command, wherein the control command generator converts the program produced in a predetermined language, and then optimizes the converted control command.

According to a fourth aspect of the present invention, there is provided a parallel reaction execution planning design apparatus for designing a parallel reaction execution planning for executing parallel reactions by chemical reaction of a molecule, the apparatus comprising: a variable and a constant defined by associating the molecule with an encoded molecule that has been encoded based on a nuclear acid sequence; a control command generator which converts a program described by a function defined to be associated with calculation reaction of the encoded molecule into a control command for driving a reaction control section which makes reaction control of chemical reaction of the molecule corresponding to the calculation reaction of the encoded molecule, and generates procedures for the control command; and an output unit which outputs the procedures for the control command, wherein the control command generator converts the program produced in a predetermined language into the control command, and then optimizes the converted control command.

According to a fifth aspect of the present invention, there is provided a parallel reaction executing method for executing a number of reactions in parallel, the method comprising: converting a program described by a function defined to be associated with a principle of parallel reactions into a control command for driving a reaction control section which makes reaction control of the parallel reactions to generate procedures for the control command; outputting the procedures for the control command to the reaction control section; and making reaction control of parallel reactions in accordance with the procedures for the control command.

According to a sixth aspect of the present invention, there is provided a parallel reaction executing method for executing parallel reactions by chemical reaction of a molecule, the method comprising: converting a variable and a constant defined by associating the molecule with an encoded molecule in accordance with a nuclear acid sequence and a program described by a function defined to be associated with calculation reaction of the encoded molecule into a control command for driving a reaction control section which makes reaction control of chemical reaction of the molecule corresponding to the calculation reaction of the encoded molecule and then optimizing the converted control command to generate procedures for the control command; outputting the procedures for the control command to the reaction control section; and making reaction control of chemical reaction of the molecule in accordance with the procedures for the control command.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram depicting a general configuration of a molecular computer device according to one embodiment of the present invention;

FIG. 2 is a view showing a specific example of the computer device according to the invention;

FIG. 3 is a view showing an allocation example of a molecular computer 22; and

FIG. 4 is a view showing a program example according to the present embodiment.

### DETAILED DESCRIPTION OF THE INVENTION

An embodiment of the present invention will be described with reference to the accompanying drawings. The term “parallel reaction” used in the invention denotes that two or more reactions corresponding to two same and/or different reaction results, respectively, are executed at the same time or in a duplicate manner. Therefore, two or more reactions configuring a parallel reaction are executed by one or more automation device designed for executing each reaction. In the case where two or more reactions are executed at the same time, mixing may be carried out in an identical reaction container or may be carried out in separate reaction containers substantially at the same time. In the case where two or more reactions are carried out in duplicate manner, although mixing is generally carried out in order planned for separate reaction containers, reaction conditions (for example, temperature and a reagent for starting reaction) which are different from each other in the same reaction container may be applied. In addition, the term “reaction” used in the invention denotes that a desired response feature relating to an arbitrary substance is checked as to whether or not the reaction feature can be obtained by a proper reactive substance. A typical reaction is a chemical reaction. Preferably, in a molecule reaction using a nuclear acid, in particular, it is possible to apply this reaction to a DNA computer which includes a molecule computing process. Chemical reaction can be applied for a variety of purposes such as refining, inspection, waste disposal, repair, assembling and the like. As a device for executing parallel reaction, publicly known robot means may be utilized for the sake of the present invention. However, when advantages of the invention are attained, such robot means can be provided as a very simple and small-sized configuration, and biological resources or environmental resources required for completing parallel reaction can also be minimized.

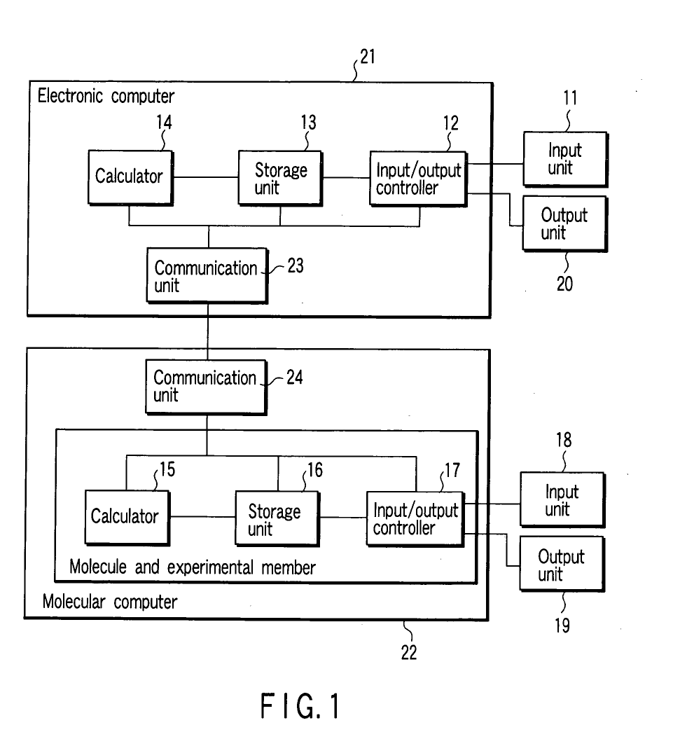

FIG. 1 is a block diagram depicting a general configuration of a molecular computer device (hereinafter, simply referred to as a “computer device”) according to one embodiment of the present invention. The computer apparatus according to one embodiment of the invention essentially comprises an electronic computer 21 and a molecular computer 22. The electronic computer 21 substantially controls a function of the molecular computer 22, and calculation is carried out by using the molecular computer 22 under the control of the electric computer section 21. The molecular computer 22 substantially executes computation by executing a chemical reaction of a molecule.

As has been described above, in the computer device according to one embodiment of the invention, the molecular computer 22 carries out calculation by using parallelism of molecule computation. Based on the data obtained as a result of such calculation, the electronic computer 21 displays the computation result. In one embodiment of the present invention, there is disclosed a computer device for efficiently executing parallel data processing or gene analysis by carrying out computation using a nuclear acid molecule. In addition, data and programs are expressed by the nuclear acid molecule, the calculation defined by the programs replaced with a molecule reaction, and reaction is executed, thereby achieving a large memory capacity and a high parallel processing capability as compared with a conventional electronic computer.

The molecular computer device according to one embodiment of the invention is provided as a computer device in which the molecular computer 22 and the electronic computer 21 complementarily share functions each other. An input unit 11 for inputting data and an output unit 20 for outputting data are connected to the electronic computer 21. In addition, an input unit 18 for inputting data and an output unit 19 are connected to the molecular computer 22.

The electronic computer 21 is achieved by a general electronic computer. Specifically, this computer section comprises a calculator 14, a storage unit 13, an input/output controller, and a communication unit 23. The storage unit 13, the input/output controller 12, and the communication unit 23 are connected to the calculator 14. Further, the input unit 11 and the output unit 20 are connected to the input/output controller 12. Furthermore, the electronic computer 21 may comprise other constituent elements of a general electronic computer. The calculator 14 is achieved by a CPU or the like, for example. The storage unit 13 is achieved by a semiconductor memory or the like.

The molecular computer 22 comprises a computer section 15; a storage unit 16, an input/output controller 17, and a communication unit 24. The computer section 15 and storage unit 16, the input/output controller 17, the input unit 18, and the output unit 19 are achieved by molecules or reactor equipment such as containers and pipetters. That is, the molecular computer 22 is achieved by a device for executing chemical reaction of a molecule, and equipment constituent elements do not correspond to the calculator 15, the storage unit 16, the input/output controller 17 and the like on a 1:1 basis. The constituent elements such as the calculator 15, the storage unit 16, and the input/output controller 17 are shown as an example of constituent elements in the case where molecules or reactor equipment such as containers and pipetters of the molecular computer 22 are categorized as the same concept as the electronic computer 21. The calculator 15, the storage unit 16, and the input/output controller 17 are connected to the communication unit 24. The input/output controller 17 is connected to the input unit 18 and the output unit 19. By these molecules and reactor equipment, calculation, data storage, and molecule data input/output control are achieved at the same time.

In the computer device according to the invention, the electronic computer 21 controls the molecular computer 22. When a desired program is inputted to the electronic computer 21, a planning of molecular computation to be executed in the molecular computer 22 is generated based on the inputted information. Further, a design is made for molecules required for the target molecular computation, and the thus obtained information is transmitted to the molecular computer 22. The molecular computer 22 carries out molecular computation based on the obtained information. For example, with respect to the molecular computation planning may be made by the calculator 14 making a search for a table in which the input information stored in the storage unit 13 and molecular computation to be designed are associated with each other, and then selecting a corresponding computation. In addition, a molecular design may be made by the calculator 14 making a search for a table in which the input information stored in the storage unit 13 and the molecules to be designed are associated with each other, and then selecting the corresponding molecule.

As the input unit 11 at the side of the electronic computer 21, there can be used any of input means by which one can input information in general, such as a keyboard and a mouse. In addition, as the output unit 22 at the side of the electronic computer 21, there can be used any of output means capable of generally outputting information, such as a display and a printer.

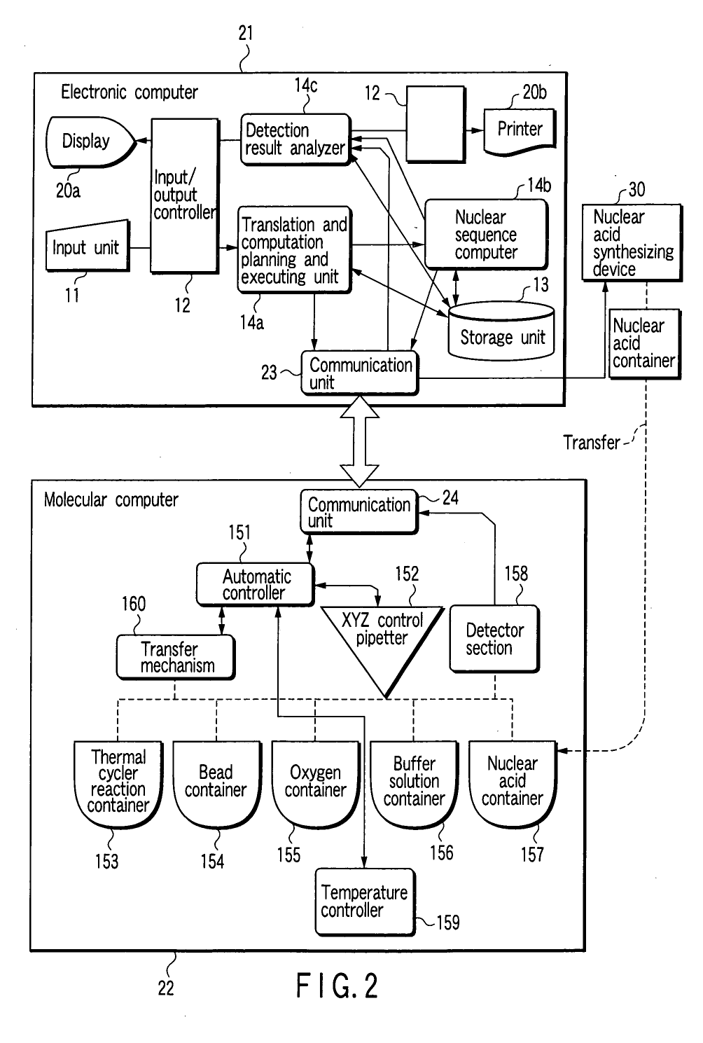

In order to achieve operations of these computer devices, although a detailed description is given later, the calculator 14, as shown in FIG. 2, functions as, for example, a translation and computation planning and executing unit 14a, a nuclear acid sequence computer section 14b, a result analyzer section 14c and the like. These sections (means) 14a to 14c are executed by a translation and computation planning and executing program, a nuclear acid sequence computer program, and a detection result analysis program being read out from the storage unit 13, for example. In addition, a program for executing a variety of functions at the calculator 14 exists as a program product recorded in a recording medium. This program is read out by a recording medium reader device (not shown), so that the program may be executed by the calculator 14 after it has been stored in the storage unit 13. The calculator 14 functions as means executed by a general electronic computer as well as the above means 14a to 14c.

Further, an output of the computation result obtained in the molecular computer 22 is transmitted to the electronic computer 21 via the communication units 24 and 23 so that desired data processing may be applied by the calculator 14 or the like. For example, these computation results are counted and calculated in a format according to a processing format stored in advance in the storage unit 13. The calculation results are finally outputted to the output unit 19 or output unit 20 in a desired mode. During these processes, the states of the calculator 15 and storage unit 16 of the molecular computer 22 are indirectly grasped as information received in the electronic computer 21 via the communication units 23 and 24.

The molecular computer 22 experimentally synthesizes nuclear acid molecules or carries out a desired calculation reaction by using the synthesized nuclear acid molecules, thereby executing molecular computation.

In addition to the processing carried out by a calculator of a general electronic computer, the calculator 14 of the electronic computer 21 converts an initial value inputted from the input unit 11 into an encoded molecule expression; converts a procedure or a function of the computer program into calculation reaction of the corresponding encoded molecule; and generates procedures for executing calculation reaction from the stored computer program. In addition, for example, the electronic computer 21 allocates a molecule to a variable; allocates a container which contains a reaction solution such as a reagent; allocates reactor equipment (such as pipette chip, for example) or reaction solution during molecular computation; moves the reactor equipment such as a container or a pipetter or allocates operations; sets temperature control of a thermal cycler; and determines a sequence for executing computation.

Now, the present invention will be described below in more detail. FIG. 2 shows a specific example of the computer device according to the invention. The computer device shown in FIG. 2 includes an electronic computer 21, a molecular computer 22, and a nuclear acid synthesizing device 30. The electronic computer 21 comprises a communication unit 23, and the molecular computer 22 comprises a communication unit 24. Data is transmitted and received between these communication units 23 and 24, thereby enabling information communication between the electronic computer 21 and the molecular computer 22. Although FIG. 1 shows an example when the nuclear acid synthesizing device is incorporated in the molecular computer 22, FIG. 2 shows that the nuclear acid synthesizing device is provided independently of the molecular computer 22.

The communication unit 23 of the electronic computer 21 is electrically connected to the nuclear acid synthesizing device 30 so that the nuclear acid synthesizing device 30 can execute nuclear acid synthesis based on a nuclear acid synthesizing command transmitted from the communication unit 23. The nuclear acid synthesizing device 30 comprises a control section which controls a nuclear acid container or a nuclear acid molecule in accordance with a command from the communication unit 23, for example. Then, the nuclear acid synthesized under the control of the control section is transferred to the molecular computer 22 while it is housed in the nuclear acid container. The nuclear synthesizing device 30 can be provided at the molecular computer 22. In this case, there is no need for providing a transfer system for transferring the container from the nuclear acid synthesizing device 30 to the molecular computer 22 independently of the molecular computer 22. The nuclear acid container may be transferred by means of a transfer mechanism or may be manually transferred.

The nuclear acid synthesizing device 30 may not be connected to the electronic computer 21. In this case, the electronic computer 21 outputs data required to nuclear acid synthesis. Based on the output result, nuclear acid synthesis may be manually carried out.

The electronic computer 21 comprises a translation and computation planning and executing unit 14a, a nuclear acid sequence calculator 14b, and a detection result analyzer 14c which are constituent elements corresponding to the calculator 14 of FIG. 1. A desired program is read out from the storage unit 13, whereby the calculator 14 functions as each of these sections 14a to 14c. Further, an input unit 11; and a display 20a and a printer 20b serving as the output unit 20 of FIG. 1 are connected to these sections 14a to 14c via an input/output controller 12.

The molecular computer 22 has an automatic controller 151, an XYZ control pipetter 152, a thermal cycler reaction container 153, a bead container 154, an oxygen container 155, a buffer solution container 156, a nuclear acid container 157, a detector 158, temperature controller 159, and a transfer mechanism 160 which function as the calculator 15, the storage unit 16, the input/output controller 17, and the input unit 18 shown in FIG. 1.

The molecular computer 22 comprises an automatic controller 151 connected to the communication unit 24. In order to automatically control the thermal cycler reaction container 151 for executing reaction of a nuclear acid; the bead container 154 for holding beads; the oxygen container 155 for holding oxygen; the buffer solution container 156 for holding a buffer solution; the nuclear acid container 157 for holding a nuclear acid; the temperature controller 159; and the transfer mechanism 160, the automatic controller 151 outputs a drive signal to each of these constituent elements (152 to 157, 159, and 160). These constituent elements (152 to 157, 159, and 160) each are driven upon the receipt of the drive signal, thereby controlling molecular reaction or reaction conditions. Therefore, these constituent elements (152 to 157, 159, and 160) each function as a reaction control section as a whole, each of which functions as a reaction control element.

A control by the automatic controller 151 is executed based on a control command transmitted from the electronic computer 21 via the communication unit 24. Any container may be used as the reaction containers 153 to 157 as long as it houses a solution containing a nuclear acid, such as a beaker, a test tube or a micro tube.

The thermal cycler reaction container 153 may be a reaction container capable of adjusting a temperature. For example, this container can be provided by combing it with a generally used thermal cycler. Further, although this example shows a case of using beads as a carrier, another carrier can be used. In this case, a proper constituent element can be provided instead of the bead container 154 or in addition to the bead container. The oxygen container 155 is provided together with the temperature controller such as a cooler in order to prevent oxygen from being deactivated. The temperature controller 159 such as a cooler or a heater may be allocated at another holding means if required.

Reaction in the thermal cycler reaction container 153 is executed along a control command. For example, a desired amount of contents is sampled from the containers 154 to 157 by means of the XYZ control pipetter 152, and then, reaction is carried out in accordance with a desired temperature condition or the like. Movement in each section is controlled by the automatic controller 151 incorporated in the molecular computer. The XYZ control pipetter 152 is controlled by the automatic controller 151, and moves in the XYZ direction and/or vertically as desired.

The transfer mechanism 160 transfers, for example, each of the containers 153 to 157 to another container housing means (not shown) or to the detector 158, in accordance with a control command from the automatic controller 151.

After reaction has completed, detection and identification of the reacted product are carried out in the detector 158. The detector 158 may be provided by either of detector means and analyzing means generally used for detecting and analyzing a nuclear acid molecule, such as an electrophoresis device, a sequencer, a chemical light emitting photometer and a fluorescent photometer.

The control commands generated by the electronic computer 21 are outputted to the communication unit 23. The communication unit 23 transmits these control commands to the molecular computer 22 via the communication unit 24. The molecular computer 22 controls constituent elements in accordance with the received control commands, thereby causing these constituent elements to execute molecular computation. The computation result is transmitted to the electronic computer 21 via the communication unit 23 by means of the communication unit 24.

The communication units 23 and 24 may be provided by wired communication means such as a cable or may be provided by wireless communication means such as a radio wave.

The nuclear acid synthesizing commands generated by the electronic computer 21 are outputted to the communication unit 23. The communication unit 23 outputs these nuclear acid synthesizing commands to the nuclear acid synthesizing device 30. The nuclear acid synthesizing device 30 controls constituent elements in accordance with the received nuclear acid synthesizing commands, thereby causing these constituent elements to execute nuclear acid synthesis. The nuclear acid container housing the nuclear acid molecule serving as the resultant product is transferred to the molecular computer 22. Of course, only the nuclear acid molecule housed in the nuclear acid container may be carried into the nuclear acid container of the molecular computer 22.

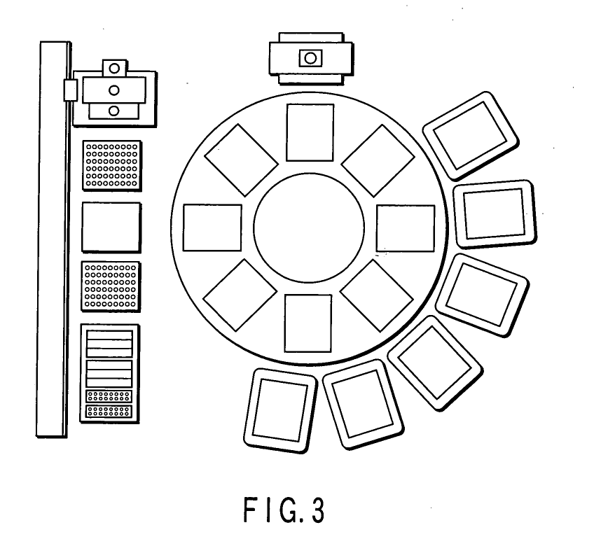

FIG. 3 is a view showing an allocation example of the molecular computer 22. A nuclear acid extracting device (not shown) is allocated adjacent to the molecular computer 22, and a transfer device (not shown) is provided partly of the nuclear acid extracting device. In one embodiment of the invention, a main object is to automatically optimizing control of reaction executed by the molecular computer having the above-described configuration. The molecular computer 22 shown in FIG. 3 will be briefly described here.

Roughly speaking, the molecular computer 22 starting from a plurality of given solutions which are “inputs” produces (a plurality of) new solutions serving as “outputs” by repeatedly applying the following operation. If this operation is deemed to be calculation, this calculation is terminated by checking these solutions which are “outputs”. In this case, for example, the following operation is made.

(1) A new solution is produced by mixing specific solutions (Mixing, suspension).

(2) A certain substance (which may be deemed to be a solution) is extracted from the solution by using magnetic beads (Magnetic separation, magtration).

(3) The solution is warmed up to a specific temperature (Warning, temperature control).

(4) The solution is maintained for a predetermined period of time at a specific temperature (Wait, maintaining).

In consideration of the compatibility with a general electronic computer, a solution can be deemed to be a “value” in a general programming language. In addition, a plate and a chip can be deemed to correspond to a “variable” for holding this “value”. A plate is a device for pouring a solution. In addition, a chip is a glass based tapered tube, and can hold a solution. In actuality, this chip includes a plurality of tubes, and is placed in a container having a plurality of holes for housing a chip called a chip rack. An operation for this chip is made for a set of these chips at the same time. The plate is also composed of a plurality of holes corresponding to a plurality of tubes, similarly, and a solution is poured in each of these holes.

Although the numbers of plates and chips are not basically limited, the molecular computer 22 shown in FIG. 3 makes the above four operations by using eight tables. In addition, two tables enable the above temperature control of (3). Further, only one device called IMU is provided for carrying out suspension of (1) and magtration of (2). The essential modules of the molecular computer 22 shown in FIG. 3 are as follows.
(1) Integrated magnetic separation unit (IMU)
(2) Stocker
(3) Dispenser
(4) Turn table
(5) Robot arm

In the above configuration, the turn table is provided as a rotary device comprising eight tables (two of which are temperature controllable). One of the eight tables is allocated immediately beneath the IMU. The IMU can pack a chip and can discharge a solution from the tapered lower part of the chip or charge it. In addition, in the IMU, an electromagnet capable of making ON/OFF operation is allocated downwardly of the turn tables, so that magnetic particles can be selectively caused to adhere to the bottom part of each hole of the plate. For example, a suspension operation is made as follows. While the IMU packs the chip, and a solution other than the chip is contained in a plate placed on a table beneath the IMP, the charge and discharge of the solution in the chip are repeated. Magtration is made similarly. Processing for causing beads to adhere to the bottom part of each hole of the plate by the IMU applying magnetization while the beads are contained in the solution is carried out. The stocker is provided as a device for providing a plate or a chip rack, and a dispenser is provided as a device for providing (dispensing) a solution. In addition, the robot arm is provided as a device for moving the plate or chip placed on one table to another table. Here, the IMU may be another technique for carrying out magnetic separation. For example, magtration can also be achieved by charging a suspension liquid into the above pipette chip, followed by causing the magnetic particles to magnetically adhere to a wall surface of the chip to discharge the solution.

In such a configuration, the eight tables each correspond to a “register” of a CPU. A “value” which corresponds to a solution is held in a plate or a chip which are “variables”. In order for the value and variables to be targeted for “calculation”, it is required to allocate them to any of the eight tables. One table can discard the held “variable” and hold another variable after the work has been completed. In addition, although a temperature control is a time consuming command, it does not require the IMU, thus making it possible for the IMU to do another work during such temperature control.

Optimization of a program considering these matters is none other than so-called register allocation and command scheduling. However, there is no example in which such optimization has been carried out for the molecular computer 22, and programming consists of direct commands to the main modules described above. Thus, in order for the molecular computer 22 to carry out reaction (calculation), a user must make a planning and describe individual reaction or operation of each section, thus requiring much expense in time and effort. For example, in the case where an incorrect program is produced, there is a danger that the molecular computer 22 is broken. Therefore, in order to describe only a required reaction and make automated operation, the following items are required.
(1) No worrying about specific table number
(2) Automated discarding of unnecessary plate or chip
(3) No worrying about a time related constraint with respect to temperature control
(4) Possible utilization of parallelism of modules
(5) Detection and elimination of inhibited operation (inhibited items)

In order to meet the above requirements, according to the present embodiment, the translation and computation planning and executing unit 14a has the following functions.

(1) Parser

A user carries out programming in a higher level language which can be described at a predetermined degree of abstract and a parser checks its correctness in the meaning, and then, detects the inhibited items. Then, the parser outputs a code for the molecular computer 22, which is correct, but is not optimized. This code is provided as a code to which register (table) allocation and optimal command scheduling are not applied yet.

(2) Code Generator

A code generator uses the code outputted by the parser as an input, carries out optimal register (table) allocation and command scheduling, and generates a final command for the molecular computer 22.

In the above-described configuration, the code generator defines a higher level language for describing a problem which includes table allocation and command scheduling at a general degree of abstract which does not depend on the molecular computer 22 (hereinafter, referred to as an “assembler language” to avoid confusion). In addition, the code generator is packed, thereby making it possible to automatically optimize scheduling. As a technique for optimizing allocation and scheduling at the same time, in the present embodiment, there is a technique based on an integer linear programming (ILP) which will be described later in detail. Such techniques based on the ILP include OASIS of Gebotys et al. and SILP of Zhang et al. These existing formulation and implementation are targeted for a general computer, and a command set is targeted for a fixed set similar to RTL. On the other hand, the present embodiment is targeted for an assembler language having the degree of abstract which enables allocation and scheduling. Therefore, the present invention is deemed to have a wide scope of application.

In the assembler language according to the embodiment, each command has a form of parallel assignments to its argument registers. In the existing technique, it has been necessary to meet a condition that only one register is assigned by a command. Therefore, in this embodiment, parallel assignments can be handled by utilizing SSA conversion. In addition, from this fact, it becomes possible to integrally express all resources as registers.

(Definition of Assembler Language)

Assuming that only a table is a register, an assembler language is defined for basic commands.

(1) newplate(t), newrack(t)

This command places a new plate and a chip rack on table “t”

(2) attach(t), detach(t)

This command attaches and detaches a chip on table “t” from and to IMU.

(3) warm(t), wait(t)

This command makes temperature control of table “t”.

(4) mix(t), mag(t)

The chip attached to IMU is suspended and magnetically separated in solution of table “t”.

(5) move(t1, t2)

Solution on table “t1” is moved to table “t2”.

This assembler language is simplified for the sake of clarity. For example, in the temperature control command of (3), although it is necessary to specify a temperature, this necessity is not essential, and thus, is simplified in the following discussion. For example, if warning up to 30 degrees is carried out, although it is necessary to specify a value “30”, this value is not defined as a command parameter, and is discriminated as warm30(t) by a command code. That is, an argument is limited to a variable which represents a real register (hereinafter, referred to as a “virtual register ”). In addition, although information on a command executing time is not described, this information is assumed to have been provided to the code generator by using any method.

A program example using the assembler language defined as described above is shown below. Here, assume that a (dispensed) plate containing a solution is placed on table t0.
warm(t1) Warming up t1 (A long time is required.)
move(t0, t1) Moving dispensed plate from t0 to t1
Wait(t1) Maintaining t1 at a specific temperature (A long time is required.)
move(t0, t1) Moving product from t1 to t2
mag(t3) Magnetically separating t3 (A long time is required.)
mix(t2) Suspending t2 (A long time is required.)

In response to the commands described above, the code generator allocates a real register (table), schedules the commands so as to be executed in parallel (for example, warm(T3) and mag(T2) described below)), and outputs, for example, the following assembler codes. In the following assembler codes, laterally arranged commands are executed in parallel. In this case, real register T1 is allocated to virtual registers t0 and t2, T3 is allocated to t1, and T2 is allocated to t3. The arguments of “warm” and “wait” are selected by T3 and T7 which are temperature controllable.
warm(T3) mag(T2)
move(T1, T3)
wait(T3)
move(T3, T1)
   mix(T1)

This assembler language is intuitive and understandable. In the case where the code generator allocates a real register, it is necessary to analyze a living range of the register. In the above-described expression, knowledge about each command is required. This knowledge is defined as follows. For example, only register numbers 3 and 7 included in the eight tables can be allocated as registers (tables) used for temperature control; “mix” and “mag” each require IMU; only IMU exists; and then, these commands cannot be executed in parallel. Further, this assembler language lacks general purpose use and expandability because a structure of the code generator depends on a specific configuration (for example, AP_{—}96 available from Olympus).

In a general compiler, a command set is predetermined, and its meaning is strictly defined. Processing from an optimization phase to the code generation is carried out based on such knowledge. Because this processing is targeted for a general computer, a wide range of commands can be expressed according to a combination of commands defined in advance, and thus, an approach that the processing system knows the knowledge about commands is proper. However, in the molecular computer 22 which is the subject of the present embodiment, it is impossible to presume basic operation such as four fundamental operations in advance. The following items are deemed to be required for the assembler language, for generating codes in such a situation.
(1) No command defines its meaning in advance.
(2) Data dependency between arguments of each command can be expressed.
(3) A set of registers which can be allocated to arguments of each command can be specified.
(4) The execution time of each command can be expressed.
(5) The possibility of parallel execution of each command can be expressed.
   (General Purpose Assembler Language)

A general purpose assembler language is derived such that the above requirements are met. First, let us consider the following command “foo” described in C language. The type of argument is considered to represent a set of registers which can be allocated. For example, this type is described by int ={i1, i2} or the like.
Command “foo” (long X, int y, int z)
Meaning X+=y*x++;

When this is expressed in the form of parallel assignment of pure (side effect free) functions foo_{—}1, foo_{—}2 into “x” and “z”, the following result is obtained:

```
/*Constraint on register set*/
x ∈ long, y ∈ int, x ∈ int
/*Parallel assignment*/
x = foo_1(x, y, z)
z = foo_2(z)
/*Function definition*/
long foo_1(long x, int y, int z) {
    return x+y*z;
}
int foo_2(int z) {
    return z+1;
}
```

In view of the code generator according to this embodiment, only a register set of arguments and argument data dependency may be present. Since foo_{—}1, foo_{—}2 are side effect free functions, even if no detail thereof exists, argument data dependency can be determined. Therefore, the information excluding definitions of these functions is deemed to be “type” of “foo”, and is described as follows.
foo: (x:long, y:int, z:int), x=(x, y, z), z=(z)

From only this description, data dependency of foo is determined.

Generally, type declaration of operating code “op” is as follows.

$$op \text{:} ( {v}_{1} \text{:} {T}_{1} , {v}_{2} \text{:} {T}_{2} , … , {v}_{n} \text{:} {T}_{n} ) , {v}_{1} = ( {v}_{i} , … )$$

$${v}_{2} = ( {v}_{j} , … )$$

$${v}_{n} = ( {v}_{k} , … )$$

where vi:Ti denotes that a certain element of set T1 of real registers is allocated to virtual register “vi”. In addition, vi=(vj, . . ) denotes that “vi” depends on (vj, . . . ). According to the previous discussion, this vi=(vj, . . . ) is obtained by eliminating function “op_i” from vi=op_i (vj, . . . ). If a definition of op_i is given, it is possible to determine how a value of an argument register is converted by executing a command as well as the fact that “vi” merely depends on (vj, . . . ). That is, when the values of virtual registers vi, vj and vk are vi0, vj0 and vk0, respectively, and a relationship of vi=(vj, vk) is established in type of “op”, a value of virtual register “vi” is obtained as op_{—}1 (vi0, vj0) by executing a command “op(v1, . . . , vi, . . . , vn). Even if a definition of function “op_i” is not given, it is possible to execute a command, assuming that such failure to define the function is regarded as a mere constructor, and to represent a value of a specific virtual register at a specific point in the form of composition of constructor.

The semantics of an assembler program according to the embodiment is defined utilizing the above fact. The meaning of the assembler program is as follows. That is, assuming that the value of each virtual register “v” at the time of starting a program is v0, a program is executed in the above meaning. After executing the last command, the semantic is the list of values of the virtual registers of the argument of the command.

For example, in the above example of “foo”, the following program is executed.
foo(x, y, z)
foo(x, y, z)

Then, the list of values of x, y and z after the execution is as follows.
foo_{—}1(foo_(x0,y0,z0),y0,foo_{—}2(z0))
y0,
foo_{—}2(foo_{—}2(z0))

The above required items (1) to (3) for the assembler language are met. In addition, with respect to required item (4), the execution time of each command may be specified. Expression (5) of the possibility of parallel execution of each command can be achieved by representing a finite number of execution units (eight units in this embodiment) as registers. A detailed description will be given below.

(Expression of Device and Constraints by Registers)

For example, the declaration for the use of such ALUs is specified at the beginning the command as follows.
/*Register set*/
ALU={A1, A2}
/*Type declaration*/
op: (a:ALU, . . . ).a=( )

In this manner, two calculating commands are executed in parallel. Here, a=( ) in the type of “op” means that a specific value independent of another argument is assigned to “a”. In actuality, although something is not assigned to ALU, the fact that “this command momentarily uses ALU, and nothing is left in the ALU” is expressed by this typing.

For example, in the following example, there does not occur an incorrect data dependency between OP1 and OP2.
/*Register set*/
ALU={A1, A2}
/*Type declaration*/
op1: (a:ALU, x:X,y:Y).a=( ),x=(y)
op2: (a:ALU, x:X,y:Y).a=( ),x=(y)
/*Program*/
op1(a,x,y)
op2(a,z,w)

That is, by setting a=( ), the living range of “a” of each command is only during execution of that command. Therefore, two lowercase letters “a” in OP1 and OP2 can be regarded as different virtual registers, thus making it possible to allocate the same register and to allocate A1 and A2 to a respective one of these registers, and then, cause the allocated registers to execute processing in parallel. That is, parallel processing of the two registers is carried out. Of course, the above “possibility” denotes that such an option is allowed when the code generator carries out optimization in consideration of both of allocation and scheduling.

In particular, considering a constraint which occurs in the case where ALU is the singleton {A1}, although no data dependency occurs with OP1 and OP2, as described previously, a constraint that processing cannot be executed at the same time is expressed. An irregular constraint that processing cannot be executed at the same time only when a combination of specific commands is used can be described in such a manner.

In the case where a=( ) is a=(a) in the above type, a value of “a” is obtained as op2_{—}1 (OP1_{—}1(a0)) after OP2 has been executed. Thus, the sequence of OP1 and OP2 cannot be exchanged. By utilizing this fact, a constraint on the execution sequence between two commands can be expressed by typing. The constraint on the execution sequence is naturally expressed by data dependency in general. However, this constraint can be expressed by introducing a virtual register set even if it cannot be naturally expressed.

Now, let us consider a type of an input/output device. The following “out(o, x)” expresses a command for outputting data “x” to any output device “o”.
/*Register set”/
DUT={01}
/*Type declaration*/
out: (o:OUT, x:DATA).o=(o, x)
/*Program: Lowercase letters a, b and c are
sequentially outputted*/
out(o, a)
out(o, b)
out(o, c)

Considering that an output is a simple assignment, a type of “out” may be as follows.
out: (o:OUT, x:DATA), o=(x)

In this case, however, a value of “o” in the last command is obtained as “out_{—}1(c0). That is, a constraint on the execution sequence of the previous “out” commands is eliminated, and the code generator may generate an incorrect code. Considering the fact that a next state of an output device is determined by an output operation, depending on a state before the output operation and data to be outputted, the above typing is natural.

On the other hand, the next state of an input device is determined by an input operation depending on “only” the state before the input operation. Thus, an input command has the following type.
/*Register set”/
IN={I1}
/*Type declaration*/
in: (i:IN, x:DATA).X=(i), i=(i)
   (ANP-96 Assembler Language Expression)

Under the above discussion, when IMU is explicitly indicated, a set of tables which are temperature controllable is explicitly indicated as a register set, and an example of the ANP-96 assembler language described previously is rewritten, the following result is obtained, where a number that follows a type designates an execution time (natural number).

| /*Register set*/ | |
|---|---|
| IMU = {IMU1} | |
| TBL = {T1, T2, T3, T4, T5, T6, T7, T8} | |
| T37 = {T3, T7} | |
| /*Declaration of type and execution time*/ | |
| newplate : (t:TBL).t=( ) | 1 |
| newrack : (t:TBL).t=( ) | 1 |
| attach : (t:TBL, i:IMU).t=(t), i=(t) | 1 |
| detach : (t:TBL, i:IMU).t=(t,i),i=( ) | 1 |
| warm : (t:T37).t=(t) | 10 |
| wait : (t:T37).t=(t) | 10 |
| mix : (t:TBL, i:IMU).t=(t,i),i=(t,i) | 10 |
| mag : (t:TBL, i:IMU).t=(t,i),i=(t,i) | 10 |
| move : (t1:TBL, t2:TBL).t2=(t1),t1=( ) | 1 |

| /*Program*/ | |
|---|---|
| warm(t1) | Warming up t1 (A long time is required.) |
| move(t0, t1) | Moving a dispensed plate from t0 to t1 |
| wait(t1) | Maintaining t1 at a specific temperature |
| (A long time is required.) | |
| move(t1, t2) | Moving a product from t1 to t2 |
| mag(t3, i) | Magnetically separating t3 (A long time |
| is required.) | |

mix(t2, i)Suspending t2 (A long time is required.)

Register sets TBL and T37 include common real registers. Thus, the register sets may not be “disjoint”. In newplate (newrack), a new plate (chip rack) is placed on table “t”. Values of “t” are obtained as newplate_{—}1 ( ) and newrack_{—}1 ( ), respectively, after command execution. Under an attach command, the chip housed in the chip rack on table “t” is packed on IMUi. A type of this command is obtained as t=(t) because a change that the chip is removed occurs. The chip on table “t” is packed on IMUi irrespective of the previous IMU state, and thus, the dependency i=(t) is established. An operation of returning the packed chip to table “t” is “detach”. With respect of type of this command, “t” is changed because the chip is returned to table “t”. However, since the result depends on the state of IMUi, more precisely, a state of the chip packed on the table, a relationship of t=(t, i) is established. In addition, the dependency part of the type of IMU is i=( ) because the chip is removed and an idle state is established. Table “t” of warm or wait must be T37, and a relationship of t={t} is established because a change depending on the previous state of “t” occurs. Types “mix” and “mag” are obtained as types of IMU mutual input and output.

The code generator for generating a code upon the receipt of the assembler language as described above comprises the following processing systems.

(1) Input

Type and Execution Time of Each Command

An assembler command line in which an argument of each command is a virtual register, and which is not processed in parallel.

(2) Output

A set of assembler commands in which a start time is added, and a virtual register is replaced with a real register.

(3) Object

Minimizing a program execution time

(4) Presupposition

An identical real register is not allocated to virtual registers whose living ranges interfere.

A command depending on preceding commands is executed after all of the preceding commands have been terminated.

It is presumed that an arbitrary number of commands can be executed at the same time (Required explicit expression of constraint on parallelism is required.)

A description will be given with respect to implementation of a code generator having the above processing systems. Hereinafter, if a “register” is merely expressed, it denotes a real register.

It is known register allocation and command scheduling are exclusively optimized each other. For example, the following formula is deemed to be established.
d=a*b+c

When this formula is converted into an assembler of a register machine in which a load latency takes one clock, the following result is obtained.

| | | |
|---|---|---|
| 1 load | v1, a | ; v1 = a |
| 2 load | v2, b | ; v2 = a |
| 3 nop | | |
| 4 mul | v3, v1, v2 | ; v3 = v1 * v2 |
| 5 load | v4, c | ; v4 = c |
| 6 nop | | |
| 7 add | v5, v3, v4 | ; v5 = v3 + v4 |
| 8 store | v5, d | ; d = v5 |

After register RI has been allocated to virtual register vi, when command scheduling is carried out, the following result is obtained.

| | |
|---|---|
| 1 load | R1, a |
| 2 load | R2, a |
| 3 nop | |
| 4 mul | R2, R1, R2 |
| 5 load | R1, c |
| 6 nop | |
| 7 add | R2, R2, R1 |
| 8 store | R2, d |

Eight Steps and Two Registers

On the other hand, when register allocation is carried out after command scheduling, the following result is obtained.

| | |
|---|---|
| 1 load | R1, a |
| 2 load | R2, b |
| 3 load | R3, c |
| 4 mul | R4, R1, R2 |
| 5 add | R4, R4, R3 |
| 6 store | R4, d |

Six Steps, Four Registers

As has been described above, the results of optimization are different from each other depending on their application sequence. In general, the living range of registers increases due to command movement for embedding “nop”. As a result, a plenty of registers is consumed, and a possibility of register spills increases. In the former application sequence, on the other hand, a register allocator makes effort to minimize the number of registers. Consequently, this allocator produces a plenty of data dependencies, and a room for a scheduler to enable command movement is reduced.

It is known that an optimization problem considering these matters at the same time is NP_HARD, and no efficient algorithm exists. However, there have been proposed a method based on integer linear programming (ILP), a method based on constraint logic type programming and the like, and there are some successful embodiments. The implementation described below is expanded based on SILP along the embodiments of the invention.

Integer linear programming (ILP) is an optimization program as described below, where V is a set of integer variables which is called ILP variables; v E V is an object variable; C is a set of linear constraint formulas. The linear constraint formula is a formula which compares linear combination of V with integer coefficient V by “=” or “≦”.
ILP=(V,v,C)
solver:ILP→sol
sol:V→Z

A program “solver” is provided to obtain an optimal solution from a given ILP. The optimal solution denotes minimizing object variable “v” from among such assignments which all the constraint in C hold.

Now, a method for expressing the code generator by ILP will be described below. First, an input program is given by the required conversion or preprocessing in the following manner.
Prog=Sequence of assembler commands
Inst=Index of Prog
Vreg=A set of virtual registers
Rset=A set of register sets
rset: Vreg→Rset
dura: Inst→N
dep={(i, j) ∈ Inst×Inst | “j” depends on “i”}
def: Vreg→Inst
refs Vreg→ [symbol] (Inst)

Hereinafter, a command “i ∈ Inst” denotes an i-th command among “Preg”. The set “Vreg” is a set of virtual registers which appear in a program. In the example of ANP_{—}96 described previously, a relationship of Vreg={t0, t1, t2, t3, i} is established. The set “Rset” is a set of register sets. In the previous example, a relationship of Rset={{IMU1}, {T1, T2, . . . , T8}, {T3, T7}} is established. The function “rset” is a function for associating a register set which can be allocated to a virtual register, and this function is determined from a command set declaration. A function “dura” is a command execution time. A function “dep” is a (true) dependency which occurs in the case where command “j” refers to the virtual register written by command “i”, and this function is obtained from type declaration and program analysis.

Further, assume that an input program is allocated in an SSA format. There are two reasons for this assumption. One of the reasons is that a pseudo dependency such as reverse dependency or output dependency is eliminated, and a room for optimization increases. From this assumption, the function “dep” considers a true dependency. The other reason, which is more large, the assumption of SSA is necessary in ILP conversion described later. In actual implementation, SSA conversion is made as preprocessing targeted for a given program. By this assumption, a command “def(v)” for defining a virtual register “v” is uniquely defined. refs(v) denotes the commands that refer to “v”.

In response to the above inputs, when a set “Vb” of ILP variables representing a start time point of each command is introduced, the ILP is a problem to minimize the number of execution steps as follows.
ILP=(V, step, C)
V=Vb∪V₁∪Vᵣ
V_{b}={b_i|i∈Inst}
C=Cₛₜₑₚ∪C_{sch}∪C_{reg}
Cₛₜₑₚ={0≦b_i+dura(i)≦step|i∈Inst}

A constraints “Csch” is provided for command scheduling, and a constraint “Creg” is provided for register allocation. Under these constraints, a solution is obtained such that the number of steps is minimized by means of an ILP solver. The ILP variables other than Vb will be described later. Hereinafter, the ILP variables are thus represented by typewriter font. In addition, an expression V_x or the like represents an ILP variable produced by converting an italic variable portion “x” in accordance with an appropriate method.

The constraint “Csch” on command scheduling can be naturally expressed by ILP as described below.
C_{sch}={b_j−b_i≧dura(i)|(I,j)∈dep}

Now, a constraint on register allocation will be described here. In the assembler language according to the embodiment, the register sets may not be “disjoint”. However, as described later, in order to express the constraint on register allocation, it is desirable that the register sets be “disjoint” each other, and instead, possible register sets be associated with each virtual register “v”. Therefore, a set of register sets “Rset” and sets “Rsets” and function “rsets” which meet the following condition, are introduced, the functions being produced from the corresponding function “rset” from a virtual register to a real register.
∪Rsets=∪Rset
x,y∈Rsets→x∩y=0
resets: Vreg→ [symbol] (Inst)
∈rsets(v)=rset(v)

These sets “Rsets” and function “rsets” which meet this condition are not unique. For example, although discrimination may be made for Rsets={{r}lr E Rset}, it is desirable that minimum division be made in order to efficiently obtain a solution of ILP. Such sets “Rsets” are obtained in accordance with the algorism below.
Rsets :={URset}
Foreach s∈Rset
Rsets :=∪{{x∩s,x\s}|x∈Rsets}

That is, starting from the largest petition, required partitions are applied. When the sets “Rsets” are obtained, the function “rsets” is defined as follows.
rsets(v)={s∈Rsets|∃x∈rset(v)x∈s}

Hereinafter, a register set denotes an element of “Rsets”. Further, a register set of virtual registers “vv” is an element in “rsets(v)”.

The register related constraints “Creg” should express the following items.
(1) Allocation exceeding the number #r of elements in each register set “v ∈ Rsets” cannot be carried out.
(2) Only one of the elements of that register set is allocated to each virtual register.
(3) Different registers are allocated to two virtual registers whose living ranges interfere.

In a graph coloring technique or the like, although allocation is carried out based on a result of living range analysis, such analysis cannot be carried out in advance because optimization of scheduling is carried out at the same time. Let us consider that a constraint excluding item (3) is expressed. Then, the constraints on only items (1) and (2) can be expressed as follows as a routing problem with a graph which is a typical applied example of ILP.

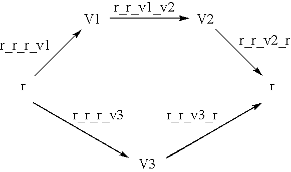

In the above expression, “vi” is a virtual register when “v” is defined as a register set, and an edge represents a register delivery. For example, an edge “r→v” denotes that one register is removed from a register set “r”, and the removed register is allocated to “vi”. An edge “v1→v2” denotes that the register used by v1 is then allocated to “v2”. In addition, an edge “v2→r” denotes that the register is returned to “r”. ILP variables which take the following boolean value (0 or 1) are provided.
{x_r_x_y|x,y∈{r,v1,v2,v3}Λx≠y}

When that value is 1, if a graph is expressed, assuming that an edge “x →y” exists, the above item (1) can be described under a linear constraint that a sum of ILP variables representing edges outgoing from “r” is equal to or smaller than #r. Item (2) can be described under a linear constraint that a sum of edges incoming to each virtual register “vi” and a sum of edges outgoing therefrom are both 1.

Now, a detailed description including expression of item (3) will be given below. First, the following ILP variables are newly introduced.
V₁={l_v|v∈Vreg}
Vᵣ=Vᵣᵥᵥ∪Vᵣᵣᵥ∪Vᵣᵥᵣ
Vᵣᵥᵥ={r_r_vᵢ_{_{—}}vⱼ|r∈RsetsΛvᵢ, vⱼ∈Vreg Λr∈rsets (vᵢ) Λr∈rsets(vⱼ}
Vᵣᵥᵥ={r_r_r_v|r∈RsetsΛv∈Vreg Λr∈rsets(v)}
Vᵣᵥᵣ={r_r_v_r|r∈RsetsΛv∈vreg Λr∈rsets(v)}

“v1” used here is a set of ILP variables which represent the living range length of each virtual register. “Vr” expresses an edge of the graph described previously. For the sake of convenience, this set is defined as a sum set of edges (Vrvv) between virtual registers; edges (Vrrv) outgoing from a register set; and edges (Vrvr) incoming to the register set. The register allocation constraint “Greg” is described by dividing it into Creg1 to Greg6 for the sake of convenience.
C_{reg}=C_{reg1}∪C_{reg2}∪ . . . ∪C_{reg6}
C_{reg1}={0≦v≦1|v^{∈}Vr}
C_{reg2}={b_j−b_i≦1_v|v∈Vreg Λi=def(v) Λj ∈refs(v)}

“Creg1” restricts a value of “Vr” to a boolean value. “Creg2” is a constraint on the living range “V1”. Here, the following auxiliary functions are defined.
outr: Rsets→ [symbol] (Vᵣᵣᵥ)
outr(r)={r_r′_r′_v∈Vᵣᵣᵥ|r′=r}
outv: Vreg→ [symbol] (Vᵣᵣᵥ∪Vᵣᵥᵣ)
outv(v)={r_r′_r′_v∈Vᵣᵣᵥ∪Vᵣᵥᵣ|v′=v}
outvr=Vreg×Rsets× [symbol] (Vᵣᵥᵥ∪Vᵣᵥᵣ)
outvr(v,r)={r_r′_v′_x∈Vᵣᵥᵥ∪Vᵣᵥᵣ|r′=rΛv′=v}
invr=Vreg×Rsets→ [symbol] (Vᵣᵥᵥ∪Vᵣᵣᵥ)
invr(v,r)={r_r′_x_v′∈Vᵣᵥᵥ∪Vᵣᵣᵥ|r′=rΛv′=v}

In the above expression, “outr(r)” are all edges outgoing from a register set “v”; “outv(v) are all edges outgoing from a virtual register; “outvr(v, r)” are all edges outgoing from the virtual register “v”, that correspond to the register set “r”; and “invr(v, r)” is an edge incoming to the virtual register “v”, the edge being included in the register set “r”, respectively, which are a set of ILP variables. The following is the remainder of “Creg”.
Creg3={Σoutr(r)≦#r|r∈RsetsΛ1≦#outr(r)}
Creg4={Σinvr(v,r)=Σoutvr(v,r)|v∈VregΛr∈rests(v)}
Creg5={Σoutv(v)=1| v∈Vreg}
Creg6={when(r_r_v1_v2=1)b_j−b_i≧1_i|
   r_r_v1_v2∈Vrvv
   Λi=def(v1)Λj=def(v2)
   Not Λ(I, j)∈ dep

“Creg3” used here denotes a constraint that the number of elements in the register set “v” is not exceeded. In addition, “Creg4” and “Creg5” denote constraints that only one register is allocated to each virtual register. In “Creg4”, “r ∈ rsets(v)” is a part of the essential register set (=∪resets(v)) of “v”, and thus, the values on both sides equal to that constraint formula are 0 or 1. Consequently, by “Creg6”, any one of the “v ∈ resets(v)” is allocated. The last “Creg6” denotes a constraint that, if x_r_v1_v2=1, i.e., if a register is delivered from “v1” to “v2”, a definition of “v2” must be later by a length equal to or greater than the living range of “v1” after “v1” has been defined. If (i, j) ∈ dep, that constraint is not required because it should be described as data dependency. If x_r_v1_v2=0, such a constraint is not required, and “v1” and “v2” may interfere on each other in living range. That is, it is inevitably required to “dynamically” control the constraint based on the value of the ILP variable “x_r_v1_v2”.

Now, a method for expressing a conditional formula using ILP will be described here.

An expression of “when (e=1) e>0” indicating that, only in the case where “e” is defined as an ILP variable of a boolean value, and a linear constraint formula “e=1” is met, the constraint formula “e”≧0 is valid cannot be essentially described by ILP. However, in SILP, this “coding” is carried out by the formula below:
e≧M(e−1)
where M is a very large positive constant. This formula itself is a linear constraint formula. If e=1, a relationship of e≧0 is established, and thus, the constraint becomes valid. If e=0, a relationship of c≧−M is established. If the range which can be taken by “e” can be estimated and if −M is smaller than the minimum value, this constraint does not eventually exist. “Creg6” can be estimated from the size of a program which is not optimized and the execution time of each command.

Let use consider a case in which “e” is not a pool value as extension of this technique. First, if a code for checking whether a value is negative or not by ILP is written by C, the following “negp” #define negp (x, y) y==(X<0?1:0) can be described as follows.
x≧−yM
0≦y≦1
x<(1−y)M

Similarly, the following “zerop” #define zerop (x, y) y==(x==0) can be described as follows.
neg(x, t1)
neg(−x, t2)
y=1−t1−t2
0≧y≧1

Such a method is effective to describe such a specific constraint condition which is not included in a basic frame.

The ILP generated by the above method is assigned to a solver, thereby solving a solution. In the present embodiment, NUOPT which is a commercially available ILP solver is used. In the OLP variables introduced in the embodiment, only “Vr” must be an integer variable. The execution time or the execution start time point may be real numbers, which is natural in machine control. In the NUOPT, an integer variable and a real variable can exist in a linear constraint formula.

Table 1 shows a time interval for solving a solution in Ultra Sparc1.

TABLE 1
Execution Time

| | | | | Time |
|---|---|---|---|---|
| | | | | interval |
| | Command | Variable | Constraint | (seconds) |
| a * b + c/d | 8 | 15 | 175 | 1.7 |
| a * b + c/(d − e) | 10 | 19 | 259 | 6.1 |
| a * (b + c) + d/(e − f) | 12 | 23 | 359 | 241.5 |

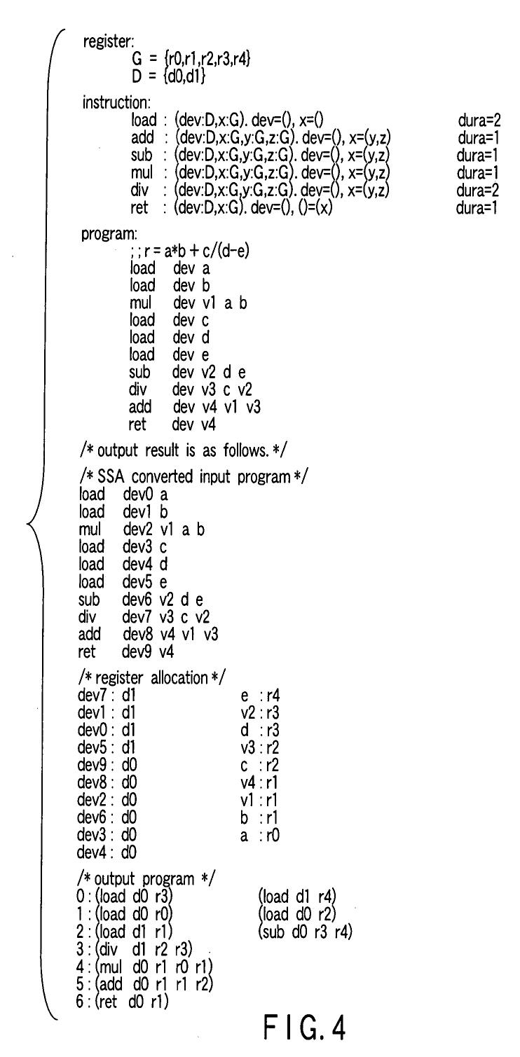

Each program denotes a result executed by replacement with the formulas at the left of the above table in the same command definition as that in FIG. 4. (The second formula is the same as that shown in FIG. 4.) In the table, a “command” denotes the number of commands produced as a result of simply compiling the formula; a “variable” denotes the number of virtual registers after SSA conversion; a “constraint” denotes the number of generated ILP constraints; and a “time interval” denotes a time interval for solving a solution. The execution time is significantly increased with a slight increase of commands or variables. It is required to improve a processing speed in order to broaden the applicability of this technique. FIG. 4 shows a program by way of example of an RISC-like assembler command with respect to a simple arithmetical formula for making easy to understand. The processing system does not have knowledge about each command as long as it is not assigned by declaration. In addition, the command execution time is two units for “load” and “div”, and one unit for other operations. A register set G represents four general purpose registers, and D represents two processing units. The argument of each command consists of: a virtual register “dev” which represents a processing unit whose command is executed; and a general purpose register for carrying out calculation.

As described above, in the embodiments of the invention, an abstractive assembler language for applying a frame of a conventional compiler technique is defined for a code generator of the molecular computer 22, and the code generator is packed. Then, in the package of the code generator, an ILP based technique is used in order to register allocation and command scheduling at the same time.

The existing researches for generating an optimized code by converting a plurality of optimization problems to ILP and solving the converted problems at the same time include: a technique for solving allocation and spill processing at the same time; techniques OASIC and SILP for solving allocation at the same time; and a technique for command selecting allocation and scheduling at the same time. If the range of programs to be handled is wide, formulation becomes complicated, and the processing time is also increased.

In the code generator according to the embodiment, command selection and spill processing are irrelative to each other. Implementation is based on SILP because of easiness of formulation. The following matters are different as compared with such formulation and implementation.

(1) A target assembler language is abstracted.

(2) An assembler command consists of a plurality of parallel assignments.

(3) All recourses are explicitly expressed as virtual registers.

(4) A common register may exist between register sets.

(5) Register allocation on a virtual register by virtual register basis.

(6) Utilization of SSA conversion.

The existing formulation and implementation are targeted for a general computer, and a command system is targeted for a fixed set similar to BT1. On the other hand, the formulation and packing according to the present embodiment are targeted for a language having a degree of abstract such that allocation and scheduling are enabled, and thus, have a wide range of application. In addition, in the existing formulation, a calculating device, a memory and the like are targeted for a general computer, and thus, are not explicitly indicated in commands. Therefore, allocation of these resources is individually formulated irrespective of processing similar to register allocation. In addition, in that formulation, there is a presumption that only one argument register is defined (assigned) by a command, and register allocation is made for a command defining a register. In the case of this embodiment, a command can express a plurality of parallel assignments, as is evident from an example of ANP_{—}96 described previously. Further, from this fact, all the resources are explicitly expressed as registers in command, and register allocation is carried out on a virtual register by virtual register basis instead of a command by command basis. In order to express this allocation by ILP, although it is required to assume that only one command defines a virtual register is required, this assumption can be achieved by making SSA conversion. Furthermore, by making such SSA conversion, there is no need for considering pseudo data dependency such as reverse dependence or output dependency which is individually supported in the conventional technique.

In the above configuration, the essential modules of the molecular computer 22 can be variously changed. These modules can be widely applied to one or more reaction executing devices or reaction planning devices such that a number of same and/or different kinds of chemical reactions are executed in parallel other than a device for carrying out molecular computation. Moreover, even a device for handling a variety of response reactions other than chemical reaction in parallel can be applied by properly associating variables, constants, and functions or the line in response reaction to the spirit of the above-described embodiments.

According to the present invention, an operation of the molecular computer can be optimized.

Additional advantages and modifications will readily occur to those skilled in the art. Therefore, the present invention in its broader aspects is not limited to the specific details, representative devices, and illustrated examples shown and described herein. Accordingly, various modifications may be made without departing from the spirit or scope of the general inventive concept as defined by the appended claims and their equivalents.

1. A parallel reaction executing apparatus comprising an electronic computer and a parallel reaction executing section, the electronic computer substantially controlling a function of the parallel reaction executing section in addition to a current computing process, and executing a number of reactions in parallel under the control thereof,
wherein control of the parallel reaction executing section by the electronic computer is executed in accordance with a program optimized after converting a higher level language produced in a predetermined language into a command of controlling the parallel reaction executing section.

2. A parallel reaction executing apparatus according to claim 1, wherein the optimization is executed based on integer linear programming (ILP).

3. A parallel reaction executing apparatus for executing reactions in parallel in accordance with chemical reaction of a molecule, the apparatus comprising an electronic computer having:
a parallel reaction executing section comprising a reaction control section which makes reaction control of chemical reaction of a molecule;
a control command generator which converts a variable and a constant defined by associating the molecule with an encoded molecule that has been encoded in accordance with a nuclear acid sequence and a program described by a function defined to be associated with calculation reaction of the encoded molecule into a control command for driving the reaction control section which makes reaction control of chemical reaction of the molecule corresponding to the calculation reaction of the encoded molecule to generate procedures for the control command; and an output unit which outputs the procedures for the control command,
wherein the control command generator converts the program produced in a predetermined language into the control command, and then optimizes the converted control command.

4. A parallel reaction executing apparatus according to claim 3, wherein the optimization is executed in accordance with integer linear programming (ILP).

5. A parallel reaction executing apparatus according to claim 3, wherein the molecule is a nuclear acid molecule.

6. A parallel reaction execution planning design apparatus for designing a parallel reaction planning for executing a number of reactions are executed in parallel, the apparatus comprising:
a control command generator which converts a program described by a function defined to be associated with a reaction principle of parallel reactions into a control command for driving a reaction control section which makes reaction control of the parallel reactions, and generates procedures for the control command; and
an output unit which outputs the procedures for the control command,
wherein the control command generator converts the program produced in a predetermined language, and then optimizes the converted control command.

7. A parallel reaction execution planning design apparatus for designing a parallel reaction execution planning for executing parallel reactions by chemical reaction of a molecule, the apparatus comprising:
a control command generator which converts a variable and a constant defined by associating the molecule with an encoded molecule that has been encoded based on a nuclear acid sequence and a program described by a function defined to be associated with calculation reaction of the encoded molecule into a control command for driving a reaction control section which makes reaction control of chemical reaction of the molecule corresponding to the calculation reaction of the encoded molecule, and generates procedures for the control command; and
an output unit which outputs the procedures for the control command,
wherein the control command generator converts the program produced in a predetermined language into the control command, and then optimizes the converted control command.

8. A parallel reaction executing method for executing a number of reactions in parallel, the method comprising:
converting a program described by a function defined to be associated with a principle of parallel reactions into a control command for driving a reaction control section which makes reaction control of the parallel reactions to generate procedures for the control command;
outputting the procedures for the control command to the reaction control section; and
making reaction control of parallel reactions in accordance with the procedures for the control command.

9. A parallel reaction executing method for executing parallel reactions by chemical reaction of a molecule, the method comprising:
converting a variable and a constant defined by associating the molecule with an encoded molecule in accordance with a nuclear acid sequence and a program described by a function defined to be associated with calculation reaction of the encoded molecule into a control command for driving a reaction control section which makes reaction control of chemical reaction of the molecule corresponding to the calculation reaction of the encoded molecule and then optimizing the converted control command to generate procedures for the control command;
outputting the procedures for the control command to the reaction control section; and
making reaction control of chemical reaction of the molecule in accordance with the procedures for the control command.
